(19) United States
(12) Patent Application Publication
(10) Pub. No.: US 2022/0323701 A1
(43) Pub. Date: Oct. 13, 2022

(54) EXPIRATION SYSTEM AND BALL JOINT FOR A PATIENT INTERFACE

(71) Applicant: Loewenstein Medical Technology S.A., Luxembourg (LU)

(72) Inventors: Martin Bechtel, Winsen/Luhe (DE); Arnold Frerichs, Buxtehude (DE); Joachim Gardein, Tenerife/Canary Islands (ES)

(21) Appl. No.: 17/658,324

(22) Filed: Apr. 7, 2022

(30) Foreign Application Priority Data
Apr. 9, 2021 (DE) 102021001834.6

(51) Int. Cl. A61M 16/06; A61M 16/08

(52) U.S. Cl. CPC A61M 16/06; A61M 16/0825; A61M 16/0858; A61M 16/0875; A61M 2016/0027

(57) ABSTRACT

Expiration system for a patient interface, comprising at least two walls, wherein the walls are arranged next to one another at least in certain sections. The expiration system has at least two flow paths for the flow of respiratory gas out of an interior of the patient interface, wherein the first flow path at least partially runs between the first wall and the second wall and the first flow path is configured for at least intermittently reducing respiratory gas pressure and wherein the second flow path is at least partially enclosed by the second wall and the second flow path is at least partially configured for the at least intermittent flow of respiratory gas into the interior of the patient interface.

### CROSS-REFERENCE TO RELATED APPLICATIONS

The present application claims priority under 35 U.S.C. § 119 of German Patent Application No. 102021001834.6, filed Apr. 9, 2021, the entire disclosure of which is expressly incorporated by reference herein.

### BACKGROUND OF THE INVENTION

### 1. Field of the Invention

The invention relates to an expiration system for a patient interface, to a patient interface and to a ball joint for a patient interface.

### 2. Discussion of Background Information

In the course of ventilation or respiratory assistance with ventilators, patient interfaces represent the link between patient/user and device. When using a single-hose system, some of the exhaled respiratory gas is breathed into the hose or the patient interface. In order to avoid an accumulation of CO2 in the respiratory gas, at least some of the respiratory gas is frequently channelled directly out of the patient interface into the surroundings. Here, the flow of the respiratory gas into the surroundings leads to noise which can be unpleasant and disturbing particularly for the user/patient. The outflowing respiratory gas can also be disturbing for any bed partner, for instance if the air impinges on any bed partner.

It would therefore be advantageous to have available a quiet and comfortable patient interface.

### SUMMARY OF THE INVENTION

The inventive expiration system for a patient interface comprises at least two walls, wherein the walls are arranged next to one another at least in certain portions. The expiration system is characterized in that the expiration system has at least two flow paths for the flow of respiratory gas out of an interior of the patient interface, wherein the first flow path at least partially runs between the first wall and the second wall and the first flow path is configured for at least intermittently reducing respiratory gas pressure and wherein the second flow path is at least partially enclosed by the second wall and the second flow path is at least partially configured for the at least intermittent flow of respiratory gas into the interior of the patient interface.

In some embodiments, the expiration system is characterized in that the expiration system comprises a third wall, wherein the third wall at least partially encloses the second flow path.

In some embodiments, the expiration system is characterized in that the second flow path at least intermittently has a branch, wherein the branch runs between the second wall and the third wall at least in certain portions coaxially to the second flow path and is designed for the at least intermittent flow of respiratory gas out of the interior of the patient interface and for at least intermittently reducing respiratory gas pressure.

In some embodiments, the expiration system is characterized in that the first flow path at least partially runs coaxially to the second flow path.

In some embodiments, the expiration system is characterized in that the first flow path, the second flow path and the branch at least partially run coaxially into one another, wherein the second flow path runs centrally and the branch at least partially runs around the second flow path and wherein the first flow path at least partially runs around the second flow path and the branch.

In some embodiments, the expiration system is characterized in that the first flow path has at least one partial flow path which runs substantially parallel to the first flow path.

In some embodiments, the expiration system is characterized in that at least one groove arrangement is arranged in at least one of the walls, wherein the groove arrangement comprises at least two grooves and wherein the grooves form channels together with the other wall.

In some embodiments, the expiration system is characterized in that the at least one groove arrangement is arranged in an inner surface of the first wall and the grooves form the channels together with an outer surface of the second wall or at least one groove arrangement is arranged in the outer surface of the second wall and the grooves form the channels together with the inner surface of the first wall.

In some embodiments, the expiration system is characterized in that the partial flow paths of the first flow path run through the channels.

In some embodiments, the expiration system is characterized in that the channels connect the interior of the patient interface to the outer region of the patient interface in a gas-conducting manner.

In some embodiments, the expiration system is characterized in that a collar is arranged on the outer surface of the second wall, wherein the collar is arranged by an underside in a form-fitting manner on an upper surface of the first wall, and wherein the underside of the collar and/or the upper surface of the first wall have/has at least one cut-out which forms at least one gap together with the upper surface of the first wall and/or the underside of the collar.

In some embodiments, the expiration system is characterized in that an extension of the first flow path runs on the underside of the collar and an extension of the partial flow paths at least partially runs in the gaps.

In some embodiments, the expiration system is characterized in that the flow cross section of the partial flow paths is not equal to the cross section of the extension.

In some embodiments, the expiration system is characterized in that the at least one cut-out on the underside of the collar extends from an outer surface of the collar to the outer surface of the second wall.

In some embodiments, the expiration system is characterized in that a collar is arranged on the outer surface of the first wall, wherein the collar has a common upper surface together with the wall.

In some embodiments, the expiration system is characterized in that at least one cut-out is formed in the upper surface of the first wall and of the collar, wherein the cut-out extends from an outer edge of the collar to the inner surface of the first wall.

In some embodiments, the expiration system is characterized in that at least one cut-out and/or gap is arranged above at least one groove arrangement and the gap is connected to the grooves in a gas-conducting manner.

In some embodiments, the expiration system is characterized in that the grooves within a groove arrangement are arranged at a uniform distance.

In some embodiments, the expiration system is characterized in that the groove arrangements are always arranged in pairs, wherein the distance between two pairs of groove arrangements is greater than the distance between the two groove arrangements of a respective pair.

In some embodiments, the expiration system is characterized in that at least three groove arrangements are arranged in the walls.

In some embodiments, the expiration system is characterized in that a groove arrangement comprises at least 2 and at most 8 grooves.

In some embodiments, the expiration system is characterized in that the number of grooves per groove arrangement is independent of one another.

In some embodiments, the expiration system is characterized in that at least 12 and at most 64 grooves are arranged in the expiration system.

In some embodiments, the expiration system is characterized in that the second wall is designed to be substantially tubular, wherein the inner surface of the second wall has a circular cross section.

In some embodiments, the expiration system is characterized in that the inner surface of the second wall constitutes a spherical sector.

In some embodiments, the expiration system is characterized in that the first wall is designed to be substantially tubular.

In some embodiments, the expiration system is characterized in that the inner surface of the first wall and the outer surface of the second wall run parallel to one another.

In some embodiments, the expiration system is characterized in that the inner surface of the first wall and the outer surface of the second wall converge conically.

In some embodiments, the expiration system is characterized in that both the inner surface of the first wall and the outer surface of the second wall have at least in certain portions a substantially circular, oval and/or polygonal cross section.

In some embodiments, the expiration system is characterized in that the second wall is part of a closure.

In some embodiments, the expiration system is characterized in that the second wall is part of the mask body.

In some embodiments, the expiration system is characterized in that the first wall is connected to the mask body via the collar.

In some embodiments, the expiration system is characterized in that the first wall forms a funnel shape together with the collar, wherein the upper surface is at an angle A to the inner surface, wherein the angle A is between 0° and 90°, preferably in a range from 22° to 75°.

In some embodiments, the expiration system is characterized in that the second wall forms a funnel shape together with the collar, wherein the underside of the collar is at an angle B to the outer surface, wherein the angle B is between 0° and 90°, preferably in a range from 22° to 75°.

In some embodiments, the expiration system is characterized in that the angle A is equal to the angle B.

In some embodiments, the expiration system is characterized in that the number of cut-outs corresponds to at least half the number of groove arrangements, but at most one more than the number of groove arrangements.

In some embodiments, the expiration system is characterized in that the grooves have a substantially rectangular cross section, optionally with rounded corners and/or a superimposed pitch circle.

In some embodiments, the expiration system is characterized in that the grooves have a maximum width of 1.25 mm and a maximum depth of 1.25 mm.

In some embodiments, the expiration system is characterized in that a notch is arranged in the inner surface, wherein a cut-out is not formed on the collar and the upper surface at the location of the notch and the notch does not represent a gas-conducting connection between an interior of the patient interface and an outer region.

In some embodiments, the expiration system is characterized in that a grip wall is arranged on the upper side of the collar at least in certain portions.

In some embodiments, the expiration system is characterized in that at least one projection is arranged on the collar, wherein a nose is arranged on the projection.

In some embodiments, the expiration system is characterized in that at least three closure pegs are arranged on the inner surface of the first wall and a corresponding number of closure slots are arranged in the outer surface of the second wall.

In some embodiments, the expiration system is characterized in that the closure pegs of the first wall are arranged irregularly and the closure slots in the second wall are arranged irregularly to the same degree, with the result that the position of the closure slots and of the closure pegs correspond in only one position of the first wall with respect to the second wall.

The patient interface is characterized in that the patient interface comprises an expiration system according to at least one of the preceding claims and a ball joint.

The ball joint for a patient interface comprises at least one ball head and a joint bearing and is characterized in that the joint head is substantially constituted by the third wall and the joint bearing is substantially formed by the inner surface of the second wall.

In some embodiments, the ball joint is characterized in that the inner surface of the second wall is designed to be spherical and the outer surface of the third wall is designed to be spherical in a manner complementary thereto, wherein the radii of curvature of the outer surface and of the inner surface are substantially equal and/or differ slightly (<1%) from one another.

In some embodiments, the ball joint is characterized in that the radii of curvature of the inner surface of the second wall and of the outer surface of the third wall are chosen such that a movement of the third wall relative to the second wall is possible.

It is to be noted that the features listed individually in the claims can be combined with one another in any technically expedient fashion and indicate further configurations of the invention. The description provides additional characterization and specification of the invention, particularly in conjunction with the figures.

It further is to be noted that an “and/or” conjunction used herein, found between two features and linking these, should always be interpreted such that only the first feature may be present in a first configuration of the subject matter of the invention, only the second feature may be present in a second configuration, and both the first and the second feature may be present in a third configuration.

A ventilator is to be understood to mean any device which assists a user or patient with natural respiration, undertakes the ventilation of the user or living being (e.g., patient and/or newborn and/or premature baby) and/or is used for respiratory therapy and/or influences the respiration of the user or patient in another way. By way of example, but without being an exhaustive list, these include CPAP and BiPAP machines, anaesthetic machines, respiratory therapy devices, (clinical, outpatient or emergency) ventilators, high-flow therapy devices and cough machines. Ventilators can also be understood to mean diagnostic devices for ventilation. Said diagnostic devices can generally be used to measure medical and/or respiration-based parameters of a living being. These also include devices which can measure and optionally process medical parameters of patients in combination with respiration or only in relation to respiration.

Unless expressly described otherwise, a patient interface can be understood to mean any peripheral device which is designed for interaction of the measurement device with a living being, in particular for therapeutic or diagnostic purposes. In particular, a patient interface can be understood to mean a mask of a ventilator or a mask connected to the ventilator. Said mask can be a full-face mask, i.e., a mask surrounding the nose and mouth, or a nasal mask, i.e., a mask only surrounding the nose. Tracheal tubes or cannulas and so-called nasal cannulas can be used as a mask or patient interface, too. In some cases, the patient interface can also be a simple mouthpiece, for example a tube, through which the living being at least exhales and/or inhales.

In the course of the invention, if an element is arranged in a surface (inner surface or outer surface), this means that this element (for example grooves) is arranged so as to be sunk in the surface. In an exemplary case of the grooves, which are arranged for example in the inner surface of a wall, the grooves are arranged in such a way that they are situated on the inner surface and project into the wall (that is to say are sunk). By contrast, elements which are arranged on the surface project. They are arranged on the surface in such a way that they are raised with respect to the wall and the surface, that is to say protrude.

### BRIEF DESCRIPTION OF THE DRAWINGS

The invention will be described in more detail and by way of example on the basis of FIGS. 1 to 12. Shown in the figures are:

FIG. 1: an exemplary embodiment of the patient interface as a mask with an exemplary embodiment of the expiration system;

FIG. 2: an exploded view of an exemplary mask body together with an expiration system and ball joint;

FIG. 3: a section through an expiration system;

FIG. 4: a greatly simplified, schematic section through an exemplary embodiment of the expiration system;

FIG. 5: an exemplary embodiment of the mask body with a first wall of the expiration system in a front view;

FIG. 6: by way of example, four regions on the upper surface of the first wall of the expiration system shown in FIG. 5;

FIG. 7: a section along the section edge X-X of FIG. 6;

FIG. 8: a detailed view of an exemplary first wall of the expiration system shown in FIG. 5;

FIG. 9: a simplified, schematic illustration of the expiration system in an exemplary embodiment;

FIG. 10: an exemplary embodiment of the first wall and of a second wall of the expiration system shown in FIG. 5;

FIG. 11: an exemplary embodiment of a closure with the second wall; and

FIG. 12: a further exemplary embodiment of a closure with the second wall.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

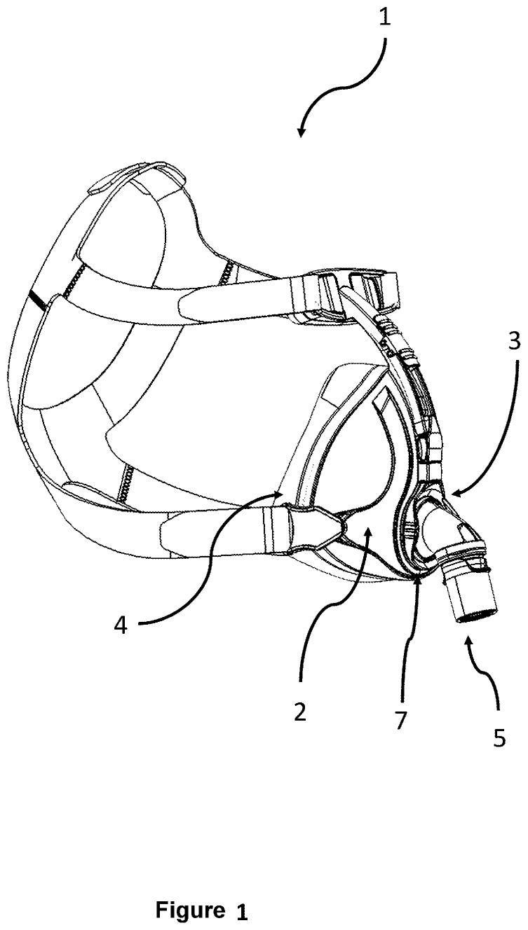

FIG. 1 shows an exemplary embodiment of the patient interface 1 as a mask with an exemplary embodiment of the expiration system 3. The patient interface 1 comprises by way of example a mask body 2 on which a forehead support and a head harness are arranged. The closure 7 forms by way of example the expiration system 3 together with parts of the mask body 2. Moreover, the closure 7 is designed by way of example such that a joint head 601 of a ball joint 6 can be received. A hose connector 5 is connected to the patient interface 1 via the ball joint 6. A mask cushion 4 is connected to the mask body 2 and is designed to have a surface lie on the face of a user/patient. Moreover, the mask cushion 4 comprises an opening which is designed to receive at least parts of the face, such as the nose and/or mouth, for example. The mask body 2 and the mask cushion 4 together enclose an interior 101 (simplified below to interior 101 of the patient interface 1) into which the face of the user/patient at least partially projects when using the mask on the face. The mask cushion 4 is for example connected to the mask body 2 via a cushion connection 204. In some embodiments, the mask cushion 4 is also part of the mask body. The interior 101 of the patient interface 1 is connected via the expiration system 3 to the outer region 102 of the patient interface 1 in a gas-conducting manner. By way of the hose connector 5, the patient interface 1 can for example be connected via a breathing hose to a ventilator in a gas-conducting manner. By way of the hose connector 5, it is thus possible, for example, for respiratory gas to be channelled or conveyed into and/or out of the interior 101 of the patient interface 1 and to the user/patient.

For example, the patient interface 1 is configured as a full-face mask, with the result that at least the nose and mouth of the user are enclosed by the mask cushion 4. However, the inventive expiration system 3 and the inventive ball joint 6 can also be used in other patient interfaces 1, such as nasal masks, tracheal cannulas/tracheal tubes and/or nasal cannulas, for example.

For example, respiratory gas is conveyed from a ventilator into the interior 101 of the patient interface 1 through the hose connector 5 and the ball joint 6 in order to assist the user/patient with breathing. For this purpose, the conveying of the respiratory gas is controlled for example via the pressure or the flow. Respiratory gas can deliberately escape continuously through the expiration system 3, as a result of which exhaled respiratory gas from the patient/user is also conveyed or washed out of the interior 101 of the patient interface 1. An accumulation of CO2 in the respiratory gas or in the interior 101 of the patient interface 1 is thus prevented and the patient/user can inhale fresh respiratory gas.

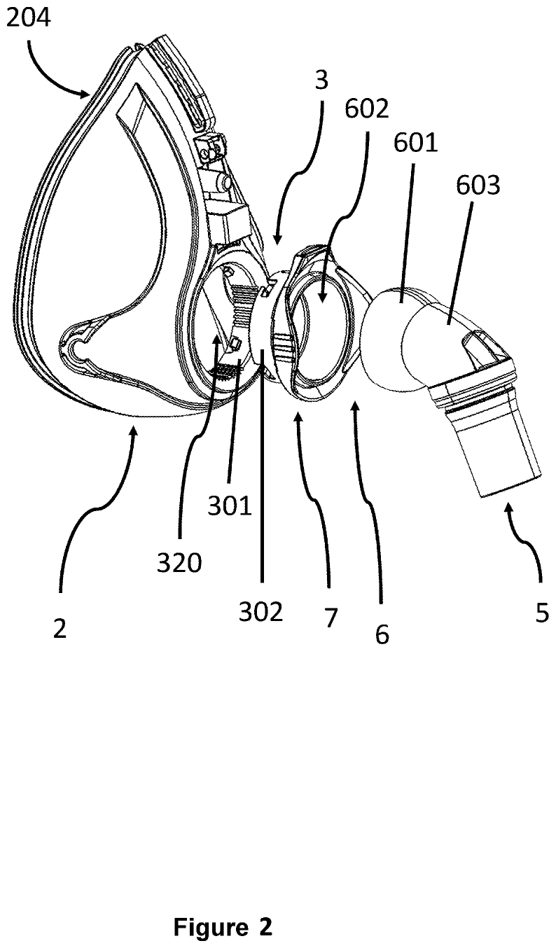

An exploded view of the mask body 2 together with the expiration system 3 and ball joint 6 is illustrated by way of example in FIG. 2. The expiration system 3 comprises by way of example at least a first wall 301 and a second wall 302. The first wall 301 is configured by way of example as part of the mask body 2 and encloses an opening 320. The opening 320 is configured and designed in such a way that respiratory gas can be conveyed to the patient and/or away from the patient through the opening 320. The opening 320 is substantially situated opposite the cushion connection 204 of the mask body 2 or the mask cushion 4. By way of example, the opening 320, and hence at least the inner surface 301a of the first wall 301, has a round cross section. In some embodiments, the opening 320 can also have a cross section which is oval and polygonal, optionally with rounded corners. A free form is also conceivable as cross section for the opening 320.

The second wall 302 is by way of example configured as part of the closure 7. The second wall 302 is arranged next to the first wall 301 on the inner surface 301a.

In some embodiments of the invention, the first wall 301 can also be configured as an extra part and be inserted into the mask body 2 and/or connected to the mask body 2, for example by encapsulation/overmoulding, adhesive bonding, welding and/or by releasable connection methods, such as plugging and/or screwing, for example.

The closure 7 also has a joint bearing 602 which is designed to receive a joint head 601. By way of example, the joint bearing 602 is configured as a hollow partial sphere, wherein the partial sphere has at least one equator C. The joint bearing 602 is formed by way of example by the inner surface 302a of the second wall 302. The joint head 601 is configured by way of example at least as a partial sphere, wherein the joint head 601 can also describe a complete sphere in some embodiments. If the joint head 601 is configured as a partial sphere, the joint head 601 thus also has at least one equator C. A tube piece 603 is arranged by way of example on the joint head 601 and is designed to be connected to a hose connector 5 or directly to a (breathing) hose. In the joint head 601 there is formed at least one lead-through, for example as a bore, so that respiratory gas can be channelled through the joint head 601. In some embodiments, the joint head is configured as a (or as part of a) hollow sphere. The wall thickness of the joint head 601 can for example also be variably configured, for example narrowing towards the openings of the lead-through and with a greater wall thickness in the region of the equator C.

By way of example, groove arrangements 303 are arranged in the first wall 301 and each comprise at least two grooves 304, preferably from four to eight grooves 304, and form channels 305 together with the second wall 302, which channels connect the interior 101 of the patient interface 1 to the outer region 102 of the patient interface 1. Here, the grooves 304 are arranged in the first wall 301 and/or second wall 302 in such a way that they at least partially interrupt the inner surface (301a, 302a) or outer surface (301b, 302b).

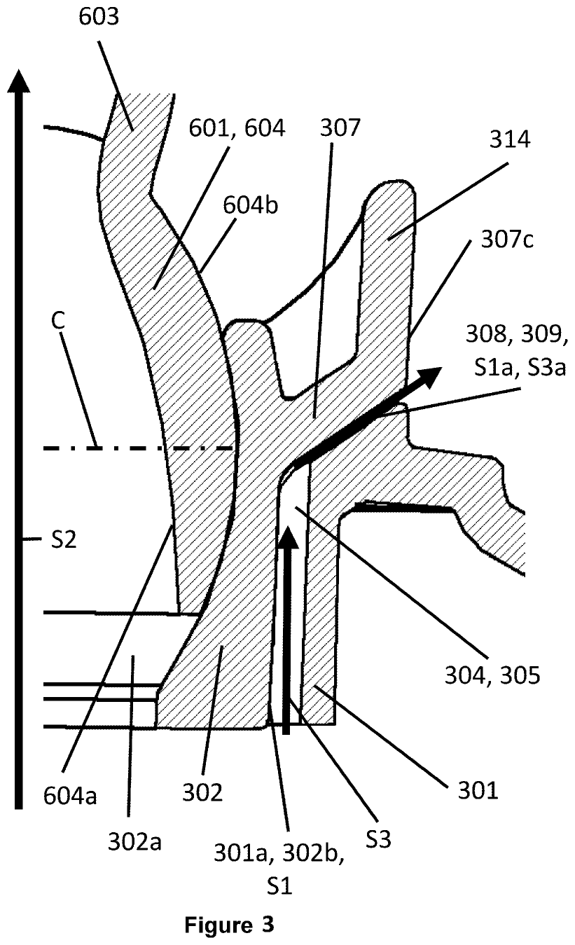

FIG. 3 shows a section through the expiration system 3, consisting of the first wall 301, the second wall 302 and the third wall 303. The second wall 302 is by way of example part of the closure 7. The third wall 604 substantially constitutes at the same time the joint head 601 of the ball joint 6. The wall 604 is by way of example substantially in the form of a spherical shell. In some embodiments, at least the outer surface 604b is designed to be substantially spherical. In some embodiments, the inner surface 604a of the third wall 604 is designed to be substantially tubular and/or has a spherical shape at least in certain portions.

A tube piece 603 is by way of example arranged on the joint head 601 or the third wall 604 and can be connected for example to a hose connector 5 or directly to a hose.

The inner surface 302a of the second wall 302 is by way of example designed, at least in certain portions, to be substantially spherical in a complementary manner to the joint head 601 or the outer surface 604b. The inner surface 302a at least partially forms the joint bearing 602. By way of example, the radii of curvature of the spherical portions of the inner surface 302a of the second wall and of the outer surface 604b of the third wall 604 are substantially equal and/or differ only slightly (<1%) from one another.

The radii of curvature of the inner surface 302a of the second wall 302 and of the outer surface 604b of the third wall 604 are by way of example chosen such that a movement of the third wall 604 relative to the second wall 302 is possible.

The first wall 301 is arranged next to the second wall 302 such that the inner surface 301a of the first wall 301 bears at least in places against the outer surface 302b of the second wall 302.

The outer surface 302b runs substantially parallel to the inner surface 301a. A gap is formed at least in certain portions between the inner surface 301a and the outer surface 302b, through which gap there runs a first flow path S1 which substantially allows a flow of respiratory gas from the interior 101 of the patient interface 1 into an outer region 102 and to a certain extent also serves at least intermittently for reducing respiratory gas pressure in the interior 101. The gap, that is to say the distance between the inner surface 301a and the outer surface 302b, is at most 0.3 mm, and preferably the distance between the inner surface 301a and the outer surface 302b is at most 0.2 mm. In certain portions, the inner surface 301a can also bear directly against the outer surface 302b, and the gap would thus be <0.05 mm.

In the exemplary embodiment shown in FIG. 3, for example a collar 306 is arranged on the outer surface 301b of the first wall 301 and a collar 307 is arranged on the outer surface 302b of the second wall 302. The common upper surface 301c of the first wall 301 and of the collar 306 is by way of example at an angle A to the inner surface 301a. The underside 307b of the collar 307 is by way of example at an angle B to the outer surface 307b of the second wall. In the exemplary embodiment, the angle A corresponds to the angle B, wherein the angle A lies in a range between 0° and 90°, preferably in a range from 22° to 75°. The underside 307b lies substantially on the upper surface 301c, wherein a gap is formed at least in certain portions between the underside 307b and upper surface 301c.

In some embodiments, the underside 307b and the upper surface 301c are formed in such a way that a gap is created at least in certain portions that constitutes an extension S la of the first flow path S1. At least in certain portions, respiratory gas can flow between the underside 307b and the upper surface 301c.

By way of example, grooves 304 are arranged so as to be sunk in the inner surface 301a of the first wall 302. The grooves 304 form channels 305 together with the outer surface 302b of the second wall 302, through which channels the partial flow paths S3 run. The partial flow paths S3 are by way of example part of the flow path S1, wherein the partial flow paths S3 run explicitly only through the channels 305. The flow path S1 thus describes by way of example the entire flow path between the first wall 301 and the second wall 302, wherein the partial flow paths S3 describe the parts of the flow path S1 which run through the channels 305.

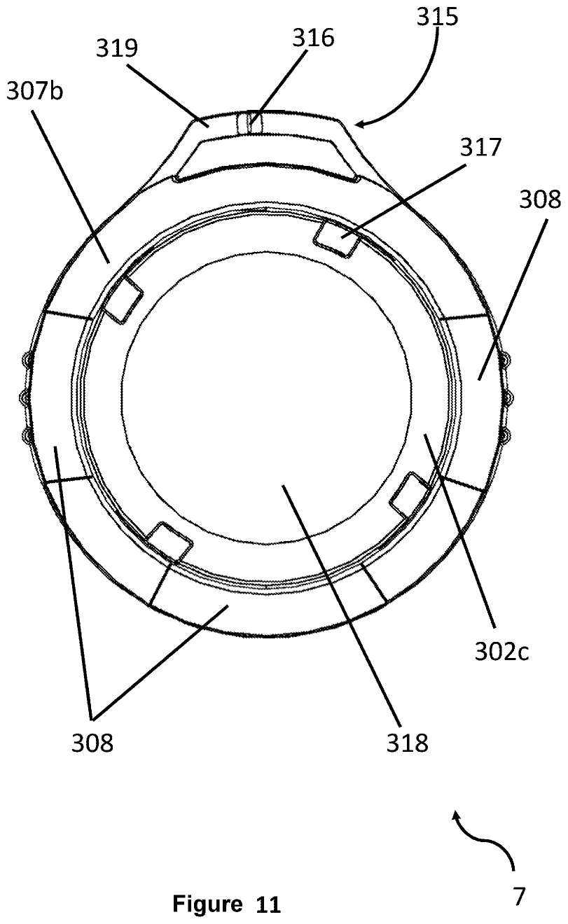

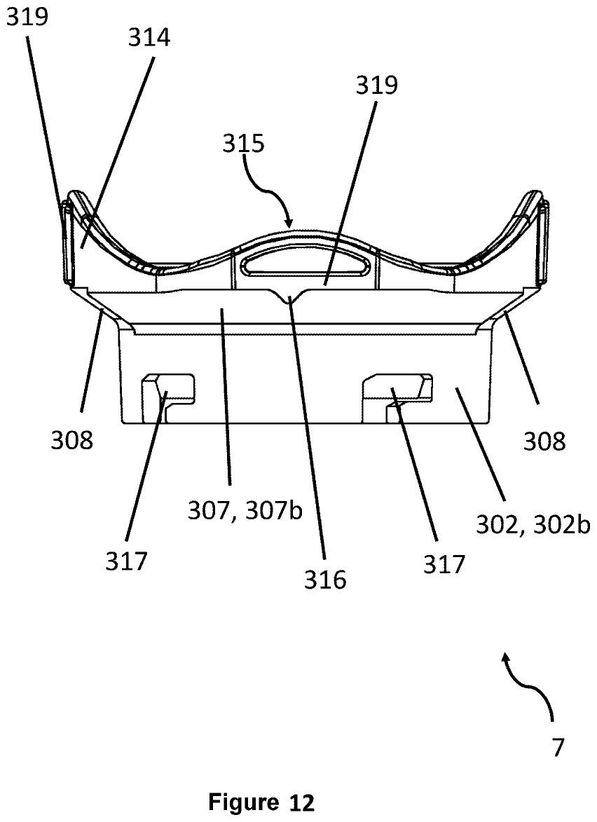

By way of example, cut-outs 308 are arranged or formed in the underside 307b of the collar 307 and form the gaps 309 together with the upper surface 301c. For example, extensions S3a of the partial flow paths S3 run through the gaps 309. In some embodiments, the flow cross section of the partial flow path S3 and the flow cross section of the extension S3a are not identical. In some embodiments, at least two, but at most 16, partial flow paths S3 transition into an extension S3a, corresponding to the arrangement of cutouts 308 in the underside 307b of the collar 307 (see also FIGS. 11 and 12). In some embodiments, the flow path S1 also transitions into the extension S3a at least in places. The gap width of the gap 309, for example dominated by the depth of the cutout 308, is between 0.1 mm and 0.8 mm, preferably between 0.25 mm and 0.45 mm. By way of example, the gap width of the gap 309 is 0.35 mm. The width of the gap 309 is for example in a range from 10 mm to 30 mm. In some embodiments, the width of the gap can increase in the direction of the outer side 307c. However, in other embodiments, the width of the gap can also remain constant. In particular, the width of the gap 309 is dependent on the positioning (base region 324, lateral region 322) and also on the number and arrangement of the channels 305. The gap 309 which is arranged in the base region 324 widens by way of example from 18 mm to 22 mm, and the gaps 309 in the lateral regions 322 widen for example from 1.5 mm to 16 mm. The length of the gap 309 lies in a range between 2 mm and 10 mm, preferably between 3 mm and 6 mm. By way of example, the length of the gap is 4.5 mm.

The flow through the partial flow path S3 makes up the majority (>95%, preferably >99%) of the total flow through the flow path S1, and the flow in the gap between the inner surface 301a and the outer surface 302b is here at most 5%, preferably at most 1%, of the total flow through the flow path 51. The flow through the extension S1a is negligibly small in comparison to the flow through the extension S3a, with, for example, the flows through S1a and S3a being in a ratio S1a:S3a of at most 1:100, with the ratio preferably being even smaller.

The third wall 604 is arranged next to the second wall 302 such that the outer surface 604b bears against the inner surface 302a at least in places. The inner surface 604a of the third wall 604 is designed to be tubular at least in certain portions, with the result that the inner surface 604a of the third wall 604 surrounds a second flow path S2. The flow cross section of the second flow path S2 is by way of example in a range from 600 mm² to 5000 mm², preferably between 700 mm² and 3500 mm², more preferably between 800 mm² and 2500 mm². In some embodiments, the flow cross section varies along the inner surface 604a, and so the flow cross section can be for example approximately 800 mm² at one side/opening and widen to 2500 mm² towards the centre. Where appropriate, the flow cross section can narrow again towards the other side/opening/transition to a tube piece 603. If the inner surface 604a of the third wall 604 is designed to be circular, the diameter can lie in a range from 14 mm to 40 mm, preferably from 15 mm to 30 mm. The flow path S2 at least intermittently allows the flow of respiratory gas out of the interior 101 of the patient interface 1. For example, it is at least intermittently possible for respiratory gas to flow out of the interior 101 via the flow path S2, for example into a connected hose. If, for example, a hose is connected—for example via a hose connector 5—to the tube piece 603, the second flow path S2 at least partially leads into the connected hose. In some embodiments, a ventilator can be connected to the patient interface 1 via the tube piece 603, together with a hose and, where appropriate, a hose connector 5, with the result that respiratory gas can also flow counter to the flow path S2 into the interior 101 of the patient interface 1 or is conveyed by the ventilator.

In some embodiments, the second flow path S2 has a branch S2a which runs through the gap between the outer surface 604b and the inner surface 302a. The branch S2a runs at least in places coaxially around the centrally running flow path S2. In some embodiments, the flow path S2 is at least in places also surrounded by the inner surface 302a of the second wall 302. The gap between the outer surface 604b and the inner surface 302a has, at least intermittently and at least in places, a width in a range from 0.01 mm to 0.3 mm, preferably between 0.05 mm and 0.22 mm. In some embodiments, respiratory gas can flow at least intermittently out of the interior 101 through the branch S2a. In some embodiments, the outer surface 604b is pressed onto the inner surface 302a at least in places as soon as a certain positive pressure builds up in the interior 101. In some embodiments, the gap between the outer surface 604b and the inner surface 302a is closed when a certain positive pressure prevails. In some embodiments, the gap width between the outer surface 604b and the inner surface 302a is reduced to such an extent that only a very small flow through the branch S2a is possible. In some embodiments, the flow between the outer surface 604b and the inner surface 302a causes the formation of a certain air cushion which, for example, allows simplified movement of the wall 604 relative to the wall 302.

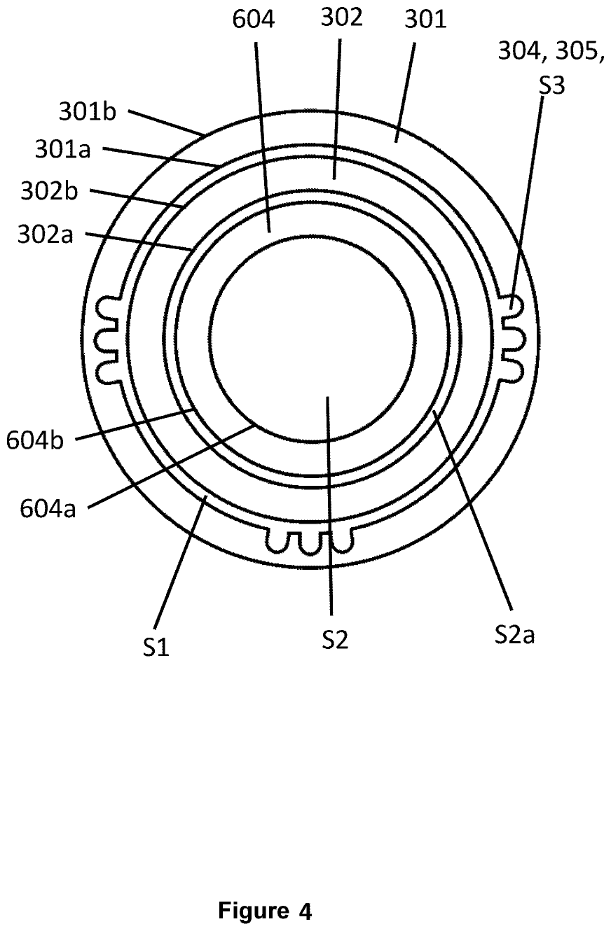

FIG. 4 shows a greatly simplified, schematic section through an exemplary embodiment of the expiration system 3. The profile of the individual flow paths and of the walls with respect to one another is intended to be illustrated by way of this figure.

The first flow path S1 runs between the first wall 301 and the second wall 302. The first wall 301 and the second wall 302 are arranged such that at least in places a gap is created between the inner surface 301a of the first wall 301 and the outer surface 302b of the second wall 302.

The first flow path S1 at least partially runs in the gap between the inner surface 301a of the first wall 301 and the outer surface 302b of the second wall 302. Grooves 304 are arranged so as to be sunk in the inner surface 301a of the first wall 301 and form the channels 305 together with the outer surface 302b of the second wall 302. Through the channels 305 runs a part of the flow path S1, the partial flow path S3. By comparison with the gap between the inner surface 301a and the outer surface 302b, the flow cross section of the partial flow path S3 is considerably larger. Overall, the partial flow path S3 thus makes up the majority (>95%, preferably >99%) of the total flow cross section of the first flow path S1.

The second flow path S2 runs substantially centrally and is surrounded by the inner surface 604a of the third wall 604 at least in certain portions. The inner surface 604a is by way of example configured to be tubular, wherein the cross section can vary along the inner surface 604a.

The second wall 302 and the third wall 604 are by way of example arranged in such a way that a gap is created at least in places between the outer surface 604b of the third wall 604 and the inner surface 302a of the second wall 302, which gap allows a movement of the third wall 604 relative to the second wall 302. A branch S2a of the second flow path S2 runs through the gap between the outer surface 604b and the inner surface 302a, wherein the flow cross section of the branch S2a comprises a fraction (<1%) of the second flow path S2.

By way of example, the branch S2a runs at least in certain portions coaxially around the centrally running second flow path S2. The first flow path S1 runs at least in certain portions coaxially around the branch S2a. The partial flow paths S3 through the channels 305 at least partially run parallel to the flow path S1 between the first wall 301 and the second wall 302.

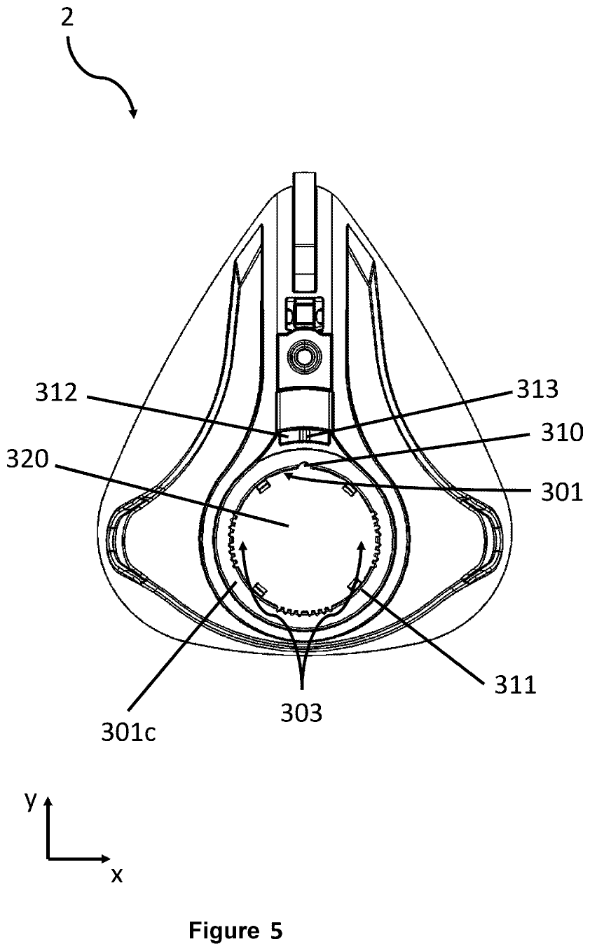

FIG. 5 shows an exemplary embodiment of the mask body 2 with the first wall 301 of the expiration system 3 in a front view. The first wall 301 surrounds the opening 320. By way of example, groove arrangements 303 are arranged, or are arranged so as to be sunk, in the inner surface 301a of the first wall 301. In the exemplary embodiment shown, a total of six groove arrangements 303 are arranged, wherein the groove arrangements 303 each comprise four grooves 304 by way of example. The groove arrangements 303 are each arranged in pairs. Here, the distance between the groove arrangements 303 within a pair is smaller than the distance of two pairs from one another. The distance between the grooves 304 within a groove arrangement is by way of example smaller than the distance between adjacent grooves 304 of two groove arrangements 303 within a pair. In some embodiments, three groove arrangements 303 are arranged in the inner surface 301a of the first wall 301, wherein the groove arrangements 303 comprise from two to eight grooves 304. The distances between the grooves 304 can vary within a groove arrangement 303. Preferably, the distances between the grooves 304 within a groove arrangement 303 are uniform and/or vary only slightly (+/−10%). Preferably, the groove arrangements 303 are not uniformly distributed around the opening 320.

By way of example, a notch 310 is formed at the position in the first wall 301 in which no grooves 304 are provided. This notch 310 does not primarily serve to form a channel 305 between the interior 101 and outer region 102 of the patient interface 1, but is designed for the production of the mask body 2 using an injection-moulding technique. In some embodiments, although the notch 310 does form a channel together with the second wall 302, no cut-out 308 is provided in the collar 307 of the closure 7 (see FIGS. 11 and 12) in order to achieve an increased flow cross section. By way of example, there is a central point here for injecting the material, with the result that a flash can occur when demolding. In order to prevent this flash being situated on the inner surface 301a of the first wall 301 and disturbing the arrangement of the second wall 302, a notch 310 is arranged in the first wall 301.

By way of example, a depression 312 is arranged above (in the y direction of FIG. 5) the opening 320 and outside the upper surface 301c of the first wall 30 By way of example, an elevation 313 is formed centrally within the depression 312. For example, the elevation 313 tapers upwards from the bottom of the depression 312, that is to say has a wider base and narrows towards an apex. By virtue of this arrangement, it is possible, for example, via a complementary nose 316 on the closure 7 (see for example FIG. 12), for there to be provided a closure mechanism which makes a rotary movement of the closure 7 within the opening 320 more difficult at least to the extent that inadvertent rotation of the closure 7 is prevented.

By way of example, four closure pegs 311 are arranged on the inner surface 301a and at least partially project into the opening 320. Together with closure slots 317, which are arranged in a corresponding manner, in the second wall 302 (see FIG. 11), there is thus realized a bayonet closure between the first wall 301 and the second wall 302 such that, after latching in the nose 316 behind the elevation 313, the closure 7 is secured against (inadvertent) release from the opening 320. Here, the closure pegs 311 are by way of example arranged at irregular distances from one another such that the closure 7 can be guided into the opening 320 only in one position. This ensures for example that the closure 7 is always inserted in the correct orientation and the channels 305 are not closed by the collar 307 of the closure 7 or only partially lie below the cutouts 308. In some embodiments, the closure pegs 311 can also be arranged at regular distances or in such a way that the closure can be inserted for example in two orientations. It is also conceivable that for example only three or five or more closure pegs 311 can be arranged on the inner surface 301a of the first wall. In some embodiments, the closure pegs 311 are arranged on the second wall 302 and the closure slots 317 are situated in the first wall 301. Moreover, other closure types are also conceivable. For example, it can also be realized that both the first wall 301 and the second wall 302 have a screw thread and the closure 7 is screwed into the opening 320. Simple plug-in connections are also possible. In some embodiments, it is also conceivable that the closure 7 is for example fixed in the opening 320 by means of a clip-lock or clamps.

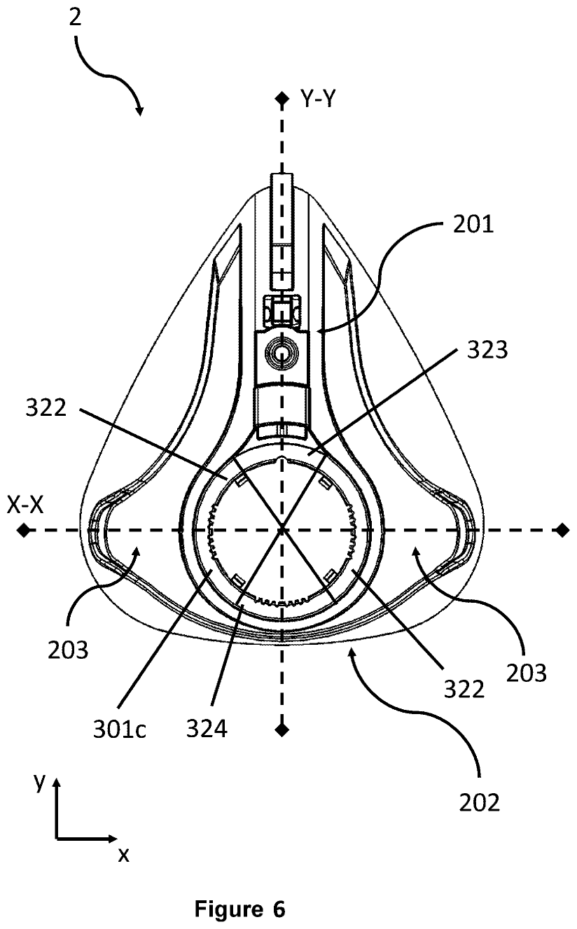

FIG. 6 depicts by way of example four regions on the upper surface 301c of the first wall 301, namely two lateral regions 322, a nose region 323 and a base region 324. These regions correspond approximately to the division of the adjoining regions (lateral region 203, nose region 201 and base region 202) of the mask body 2. By way of example, the groove arrangements 303 are arranged only in the two lateral regions 322 and/or the base region 324 of the first wall 301. The nose region 323, in which by way of example no groove arrangement 303 is provided, here assumes at most a region of 120°—in the case of a circular opening 320. In some embodiments, the nose region 323 is smaller, for example at most 90°. In some embodiments of the expiration system 3, groove arrangements 303 are arranged in all the regions 322, 323 and 324. In some embodiments, grooves can be arranged along the entire circumference of the opening 320 in the inner surface 301a of the first wall.

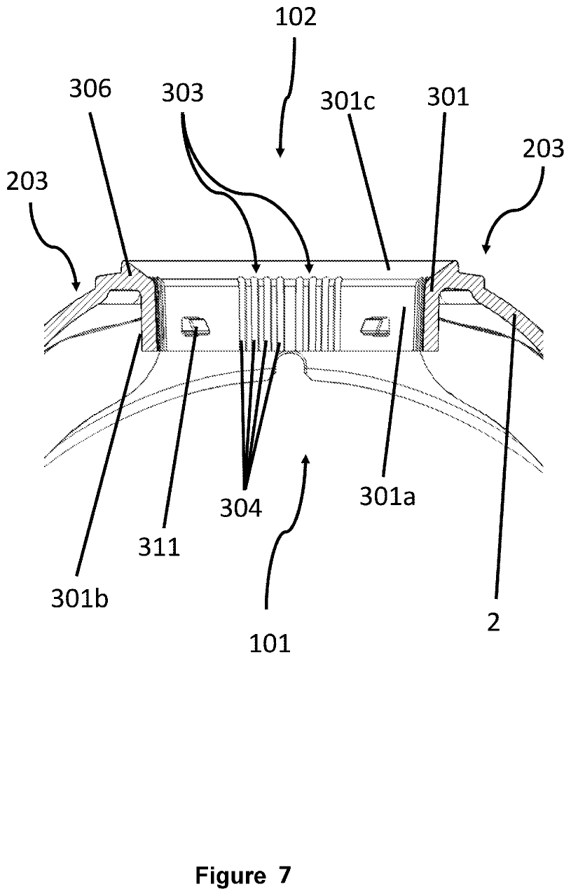

FIG. 7 is a section along the section edge X-X (FIG. 6) through an exemplary embodiment of the mask body 2, looking at the groove arrangements 303 in the base region 324 of the first wall 301.

On the inner surface 301a of the first wall 301 are arranged closure pegs 311 which are configured and designed for example to constitute a bayonet closure together with the closure slots 317 in the second wall 302. In some embodiments, the bayonet closure prevents a translational movement of the two walls 301, 302 with respect to one another, that is to say prevents the second wall 302 (as part of the closure 7) from sliding/being able to be pulled inadvertently out of the opening 320. The inadvertent or unintentional opening of the bayonet closure by rotating the closure 7 into a position in which the second wall 302 is displaceable with respect to the first wall 301 is by way of example prevented by the nose 316 latching in behind the elevation 313 (see FIGS. 5 and 11). By way of example, the closure pegs 311 are arranged such that the closure 7 can be inserted into the opening 320 in only one position and orientation.

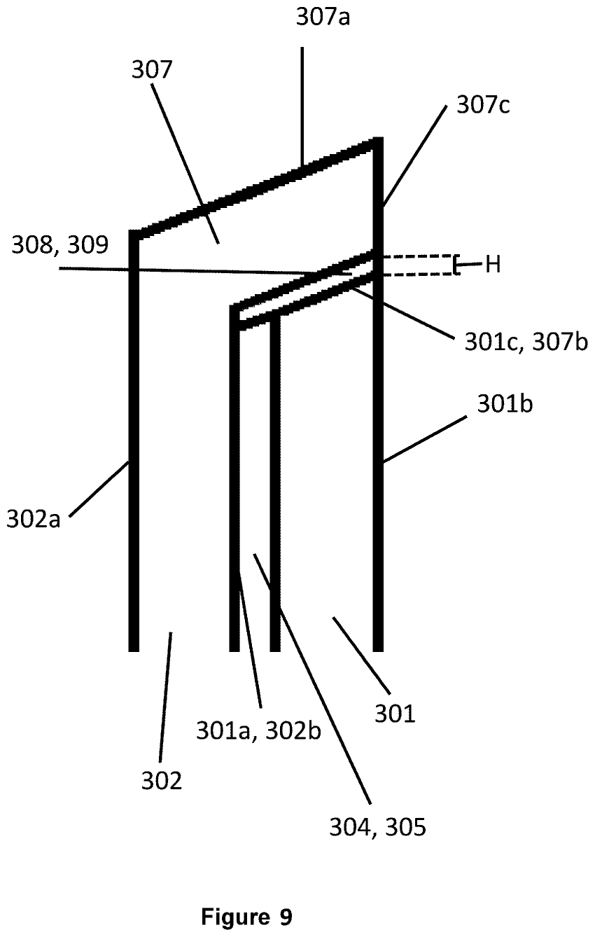

The groove arrangements 303 are arranged so as to be sunk in the inner surface 301a of the first wall 301. By way of example, three pairs of groove arrangements 303 with four grooves 304 each are arranged in the wall 301, wherein the section X-X from FIG. 6 goes through two of the pairs of groove arrangements 304. Together with the outer surface 302b of the second wall 302 (see FIGS. 9 to 12), the grooves 304 form channels 305 which connect the interior 101 of the patient interface 1 to the outer region 102 in a gas-conducting manner. Within a groove arrangement 303, the distance between the grooves 304 is uniform by way of example. The distance between the grooves 304 within a groove arrangement 303 is by way of example less than the distance between the outer grooves 304 of two adjacent groove arrangements 303. By way of example, the distance between two pairs of groove arrangements 303 is greater than the distance between the groove arrangements 303 within a pair. In some embodiments, the distance between all the grooves 304 within a pair of groove arrangements 303 is identical. In some embodiments, the grooves 304 are uniformly distributed along the first wall 301 independently of the groove arrangements 303. In some embodiments, the grooves 304 are randomly distributed independently of the groove arrangements 304. In some embodiments, the groove arrangements are not arranged in pairs and are arranged at uniform and/or random distances from one another. In some embodiments, from 24 to 64 grooves 304 are arranged along the first wall 301. In some embodiments, the distances between two grooves 304 are as wide as the grooves 304, so that, if a groove has a width of 1 mm for example, the distance from the nearest groove is likewise 1 mm. In some embodiments, the distance between two grooves 304 is independent of the width of the grooves 304.

By way of example, the grooves 304 have a length in a range between 5 mm and 20 mm, preferably between 7 mm and 12 mm, for example 8 mm (+/−5%). The width of the grooves 304 on the inner surface 301a lies by way of example in a range from 0.75 mm to 1.25 mm, preferably between 0.85 mm and 1.1 mm, more preferably between 0.92 mm and 1.05 mm. The depth of the grooves lies by way of example in a range from 0.75 mm to 1.25 mm, preferably between 0.85 mm and 1.1 mm, more preferably between 0.92 mm and 1.05 mm. The flow cross section of the channels 305 lies in a range between 0.7 mm² and 1.1 mm², preferably between 0.75 mm² and 0.9 mm², for example 0.8 mm² (+/−5%). The width of the grooves 304 can vary in the wall, in particular if the cross section of the grooves 304 is not rectangular. In some embodiments, the width of the grooves 304 on the inner surface 301a can vary, for example within a groove arrangement 303. The distance between two grooves 304 within a groove arrangement 303 lies by way of example in a range from 0.25 mm to 2 mm, preferably between 0.3 mm and 1.5 mm.

In some embodiments, at least one closure peg 311 is arranged between two pairs of groove arrangements 303.

In the exemplary embodiment of the first wall 301 that is shown in FIG. 7, a collar 306 is arranged on the outer surface 301b of the first wall 301. By way of example, the collar 306 is arranged such that it has a common upper surface 301c with the wall. By way of example, the collar 306 is the connection between the first wall 301 and the mask body 2. In the exemplary embodiment, the collar materially transitions into the mask body 2, with the first wall 301 and the collar 306 thus being integrated into the mask body 2. In some embodiments, the first wall 301 and the collar 306 are not materially connected to the mask body 2 but produced as a separate part and inserted into an opening provided therefor in the mask body 2 and connected to the mask body 2, for example via a screw connection and/or plug-in connection, by adhesive bonding and/or by welding. In some embodiments, the connection is achieved by encapsulation or overmolding. In some embodiments, the mask body 2 is also directly connected to the first wall 301, for example if no collar 306 is arranged on the first wall 301. In some embodiments, the collar 306 also runs above the mask body 2, with the result that a small gap is created between the mask body 2 and collar 306.

By way of example, the upper surface 301c is at an angle A to the inner surface 301a. Here, the angle A is between 0° and 90°, preferably in a range from 22° to 75°, more preferably in a range from 35° to 65°. In some embodiments, the angle A lies in a range from 45° to 60°. In some embodiments, the wall 301 together with the collar 306 is designed for example to be funnel-shaped, with the wall 301 or the inner surface 301a constituting the funnel neck.

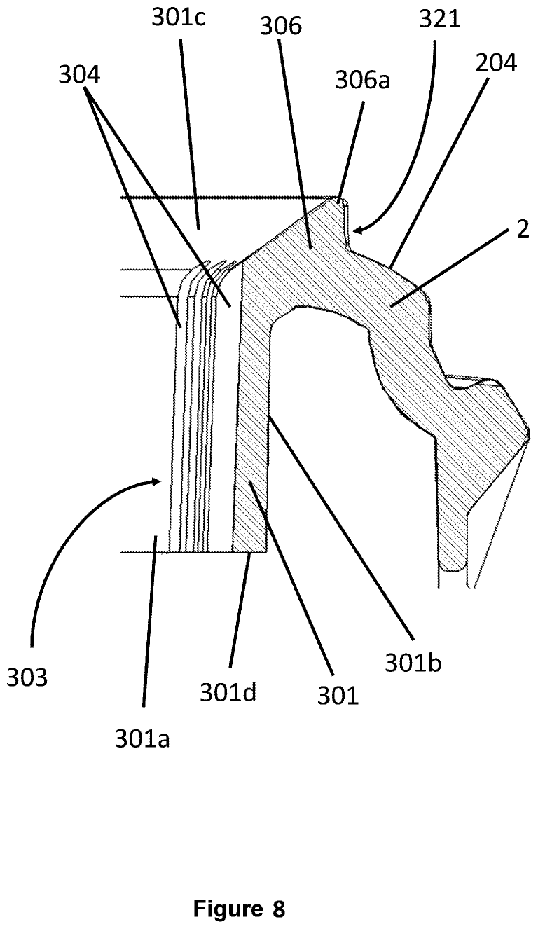

An exemplary detailed view of the first wall 301 is illustrated in FIG. 8. A section through a groove 304 is illustrated, by way of example along the section edge Y-Y from FIG. 6. The grooves 304 are arranged so as to be sunk in the inner surface 301a of the first wall 301 and run from the lower surface 301d of the first wall 301 to the upper surface 301c. Together with a second wall 302, the grooves 304 form channels 305 which connect the interior 101 of the patient interface 1 to the outer region 102 in a gas-conducting manner. The four grooves 304 shown by way of example are, for example, together a groove arrangement 303. On the outer surface 301b of the first wall 301 is arranged a collar 306 by way of example. In the embodiment shown, the first wall 301 and the collar 306 have a common upper surface 301c. The upper surface 301c is at an angle A to the inner surface 301a of the first wall 301. By way of example, the collar 306 materially transitions into the mask body 2. In some embodiments, a step 321 is formed at least in certain portions at the transition between the collar 306 and mask body 2 on the outer edge 306a. Here, this step 321 influences the flow profile of the respiratory gas in the outer region 102 or on the surface 204 of the mask body 2. The distance between the outer edge 306a and the surface 204 lies for example in a range from 0.5 mm to 4 mm, preferably in a range from 0.7 mm to 2 mm.

A simplified, schematic illustration of the expiration system 3 in an exemplary embodiment is shown in FIG. 9. The expiration system 3 substantially consists of a first wall 301 and a second wall 302 which are arranged next to one another, wherein grooves 304 are arranged in at least one of the walls 301, 302 and form channels 305 together with the respective other wall 301, 302. By way of example, in the illustrated embodiment, the grooves 304 are arranged so as to be sunk in the inner surface 301a of the first wall 301. Correspondingly, the grooves 304 form the channels 305 together with the outer surface 302b of the second wall 302. In the embodiment shown, by way of example no collar 306 is arranged on the outer surface 301b of the first wall 301. In some embodiments in which no collar 306 is arranged on the first wall 301, a step 321 is formed or arranged between the upper surface 301c of the first wall and the surface 204 of the mask body 2.

The second wall 302 is arranged with the outer surface 302b on the inner surface 301a of the first wall 301. The channels 305 are thus formed at the locations at which the inner surface 301a is interrupted by the grooves 304. On the outer surface 302b of the second wall 302 is arranged a collar 307 by way of example. The underside 307b lies by way of example on the upper surface 301c. Cut-outs 308 are arranged or formed in places on the underside 307b of the collar 307, wherein the cut-outs 308 extend from the outer side 307c or the edge between the outer side 307c and underside 307b to the outer surface 302b or the edge between the outer surface 302b and the underside 307b.

In the regions in which the cut-outs 308 are formed, the cut-outs 308, together with the upper surface 301c, form gaps 309 above the upper surface 301c. The cut-outs 308 are preferably arranged in such a way that they are arranged in the regions in which the groove arrangements 303 or the grooves 304 are also arranged. The respiratory gas, which flows out of the interior 101 of the patient interface 1 through the channels 305, is for example channelled via the gaps 309 into the outer region 102. Finally, the gaps 309 together with the channels 305 thus form the gas-conducting connection between the interior 101 and the outer region 102. In some embodiments, a cut-out 308 is formed for each groove 304. In some embodiments, a cut-out 308 is formed for each groove arrangement 304, wherein the cut-out 308 or the gap 309 extends over the entire width of the groove arrangement 303, that is to say at least from the outer edges of the outer grooves 304. In some embodiments, the cut-out 308 can also extend beyond the groove arrangement 303 by up to 10% of the entire width thereof. In some embodiments, a cut-out 308 is formed for each pair of groove arrangements 303, wherein the cut-out 308 or the gap 309 extends over the entire width of the pair, that is to say at least from the outer edge of the one groove arrangement 303 to the outer edge of the other groove arrangement 303. In some embodiments, the cut-out 308 can also extend beyond the pair of groove arrangements 303 by up to 10% of the entire width thereof.

The underside 307 is at an angle B to the outer surface 302b of the second wall 302, wherein the angle B corresponds to the angle A formed between the upper surface 301c and the inner surface 301a. The outer surface 302b of the second wall runs parallel to the inner surface 301a of the first wall. The upper surface 301c and the underside 307b run substantially parallel to one another.

The cut-outs 308 have for example a depth in a range from 0.1 mm to 3 mm. Correspondingly, the cut-outs 308 are thus formed in such a way that a gap 309 having a height H of 0.1 mm to 3 mm is created.

In some embodiments, the grooves 304 or the groove arrangements 303 are for example alternatively or additionally arranged so as to be sunk in the outer surface 302b of the second wall 302. In some embodiments, the cut-outs 308 are alternatively or additionally arranged in the upper surface 301c. If the grooves 304 or the groove arrangements 303 are arranged so as to be sunk in the outer surface 302b of the second wall 302, the statements which relate to an arrangement of the groove arrangements 303 in the first wall 301 apply in equal measure.

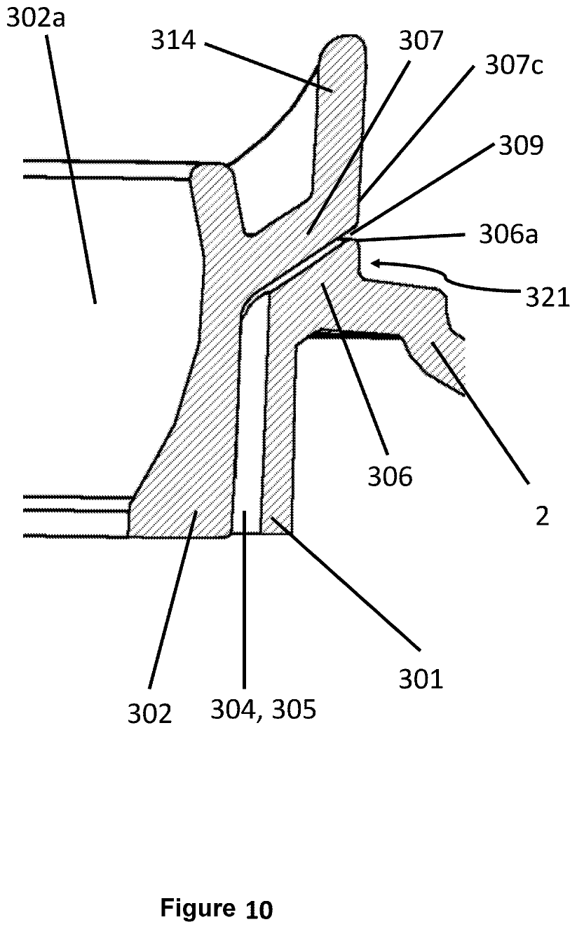

An exemplary embodiment of the first wall 301 and of the second wall 302 is illustrated in a section in FIG. 10. Here, the section substantially follows the section edge X-X from FIG. 5, with it being the case that, in addition to the mask body 2 with the first wall 301, the closure 7 with the second wall 302 is also illustrated.

By contrast with the embodiment shown in FIG. 9, the collar 306 is arranged on the outer surface 301b of the first wall 301. Between the collar 306 and the mask body 2 is arranged a step 321 by way of example. In some embodiments, the step 321 runs along the entire collar 306. In some embodiments, the step 321 does not run along the entire collar 306, but at least in the regions in which groove arrangements 303 and/or cutouts 308 are also arranged. For example, the in the nose region 323 is only partially formed.

By way of example, grooves 304 are arranged in the first wall 301 and form the channels 305 together with the second wall 302. Gaps 309 are formed by the cutouts 308 in the underside 307b of the collar 307. Respiratory gas can escape from the interior 101 of the patient interface 1 into the outer region 102 through the channels 305 and gaps 309. Arranged in certain portions on the upper side 307a of the collar 307 are grip walls 314 which by way of example are parallel to the outer surface 302b of the second wall 302.

The inner surface 302a of the second wall 302 constitutes by way of example a (hollow) partial sphere, wherein the partial sphere has at least one equator C. The inner surface 302a is designed and configured in such a way that a joint head 601 can be received. By way of example, the inner surface 302a thereby constitutes the joint bearing 602 of a ball joint. In some embodiments, the inner surface 302a is not spherical, but for example straight, optionally with fastening elements for fixing a tube. The radius of curvature of the outer surface 604b of the third wall 604 is by way of example 15.5 mm (+/−5%), but can generally be between 10 mm and 20 mm, preferably between 12.5 mm and 17.5 mm. Correspondingly, the radius of curvature of the inner surface 302a is by way of example likewise approximately 15.5 mm (+/−5%), but generally, matching the radius of curvature of the outer surface 604b, between 10 mm and 20 mm, preferably between 12.5 mm and 17.5 mm.

FIGS. 11 and 12 show an exemplary embodiment of the closure 7 with the second wall 302.

A view looking at the underside 307b of the collar 307 is illustrated in FIG. 11. The second wall 302 surrounds an opening 318 into which for example a joint head 601 can be inserted. Closure slots 317 are arranged in the outer surface 302b and the underside 302c of the second wall 302. The closure slots 317 are by way of example arranged in such a way that they correspond to the arrangement of the closure pegs 311 on the inner surface 301a of the first wall. Thus, the closure 7 can be inserted into the opening 320 in only one position or orientation.

Cut-outs 308 are arranged in the underside 307b of the collar 307 which is arranged on the outer surface 302b of the second wall 302. By way of example, three cut-outs 308 are arranged which correspond in position to the (pairs of the) groove arrangements 303 in the first wall 301. In some embodiments, a large cut-out 308 is arranged on the underside 307b of the collar 307 and extends over all the groove arrangements 303. In some embodiments of the closure 7, no cut-out 308 is arranged in the underside 307b particularly in the region of the projection 315.

By way of example, a projection 315 is arranged on the outer side 307c of the collar. On this projection is arranged a web 319 on whose underside there is formed a nose 316. This nose 316 can latch in behind the elevation 313 in the depression 312 and thus prevents unintentional rotation of the closure 7 in the opening 320.

FIG. 12 shows a side view of an exemplary embodiment of the closure 7, looking at the projection 315. In the illustration there can laterally be seen the cut-outs 308 in the underside 307b of the collar 307. No cut-out is envisaged in the underside 307b in the region of the projection 315. A web 319 with a nose 316 is arranged on the projection 315. Here, the nose 316 is by way of example not arranged in the centre of the web 319, but slightly offset such that it can latch in behind the elevation 313 arranged centrally in the depression 312.

In the outer surface 302b of the second wall 302 are arranged closure slots 317 which, together with the closure pegs 311 on the inner surface 301a of the first wall 301, result in a bayonet closure.

The outer surface 302b of the second wall 302 runs slightly conically, with the outer surface 302b running parallel to the inner surface 301a of the first wall 301. This makes it possible to achieve simpler insertion of the closure 7 into the opening 320 surrounded by the first wall 301.

By way of example, grip structures 325 are arranged on the grip walls 314 arranged on the collar 307. For example, the grip structures 325 are formed for example as elongate elevations on the grip wall 314.

### LIST OF REFERENCE SIGNS

1 Patient interface
2 Mask body
3 Expiration device
4 Mask cushion
5 Hose connector
6 Ball joint
7 Closure
101 Interior
102 Outer region
201 Nose region
202 Base region
203 Lateral region
204 Surface
301 Wall
301a Inner surface
301b Outer surface
301c Upper surface
301d Lower surface
302 Wall
302a Inner surface
302b Outer surface
303 Groove arrangement
304 Groove
305 Channel
306 Collar
306a Outer edge
307 Collar
307a Upper side
307b Underside
307c Outer side
308 Cut-out
309 Gap
310 Notch
311 Closure peg
312 Depression
313 Elevation
314 Grip wall
315 Projection
316 Nose
317 Closure slot
318 Opening
319 Web
320 Opening
321 Step
322 Lateral region
323 Nose region
324 Base region
325 Grip structure
601 Joint head
602 Joint bearing
603 Tube piece
604 Wall
604a Inner surface
604b Outer surface
A Angle
B Angle
C Equator
H Height
S1 Flow path
S1a Extension
S2 Flow path
S2a Branch
S3 Partial flow path
S3a Extension

1.-22. (canceled)

23. An expiration system for a patient interface, wherein the expiration system comprises at least a first wall and a second wall which are arranged next to one another at least in certain sections, and wherein the expiration system features at least two flow paths for a flow of respiratory gas out of an interior of the patient interface, a first flow path at least partially running between the first wall and the second wall and being configured for at least intermittently reducing respiratory gas pressure and a second flow path being at least partially enclosed by the second wall and being at least partially configured for an at least intermittent flow of respiratory gas into the interior of the patient interface.

24. The expiration system of claim 23, wherein the expiration system further comprises a third wall, which at least partially encloses the second flow path.

25. The expiration system of claim 24, wherein the second flow path at least intermittently has a branch, which runs between the second wall and the third wall at least in certain sections coaxially to the second flow path and is configured for an at least intermittent flow of respiratory gas out of the interior of the patient interface and for at least intermittently reducing respiratory gas pressure.

26. The expiration system of claim 25, wherein the first flow path, the second flow path and the branch of the second flow path at least partially run coaxially to one another, the second flow path running centrally and the branch at least partially running around the second flow path, and the first flow path at least partially running around the second flow path and the branch and having at least one partial flow path which runs substantially parallel to the first flow path.

27. The expiration system of claim 23, wherein at least one groove arrangement is arranged in at least one of the first and second walls, the groove arrangement comprising at least two grooves and the grooves forming channels together with the first and second walls.

28. The expiration system of claim 27, wherein the at least one groove arrangement is arranged in an inner surface of the first wall and the grooves form the channels together with an outer surface of the second wall or at least one groove arrangement is arranged in the outer surface of the second wall and the grooves form the channels together with the inner surface of the first wall.

29. The expiration system of claim 27, wherein partial flow paths of the first flow path run through the channels and the channels connect an interior of the patient interface to an outer region of the patient interface in a gas-conducting manner.

30. The expiration system of claim 23, wherein a collar is arranged on an outer surface of the second wall, the collar being arranged by an underside in a form-fitting manner on an upper surface of the first wall, and the underside of the collar and/or an upper surface of the first wall have/has at least one cut-out which forms at least one gap together with the upper surface of the first wall and/or the underside of the collar.

31. The expiration system of claim 30, wherein an extension of the first flow path runs on the underside of the collar and an extension of partial flow paths of the first flow path at least partially runs in the at least one gap and a flow cross section of the partial flow paths of the first flow path is not equal to a cross section of the extension of the first flow path.

32. The expiration system of claim 30, wherein the at least one cut-out on the underside of the collar extends from an outer surface of the collar to an outer surface of the second wall.

33. The expiration system of claim 23, wherein a collar is arranged on an outer surface of the first wall, the collar having a common upper surface together with the first wall, and at least one cut-out is formed in the upper surface of the first wall and of the collar, the at least one cut-out extending from an outer edge of the collar to an inner surface of the first wall.

34. The expiration system of claim 30, wherein at least one cut-out and/or gap is arranged above at least one groove arrangement and the gap is connected to the grooves in a gas-conducting manner.

35. The expiration system of claim 23, wherein at least three groove arrangements are arranged in the first and second walls, a groove arrangement comprising at least 2 and at most 8 grooves, numbers of grooves per groove arrangement being independent of one another.

36. The expiration system of claim 23, wherein the second wall is configured to be substantially tubular, an inner surface of the second wall having a circular cross section and the first wall being configured to be substantially tubular and the inner surface of the second wall constituting a spherical sector.

37. The expiration system of claim 23, wherein an inner surface of the first wall and an outer surface of the second wall run parallel to one another and converge conically.

38. The expiration system of claim 23, wherein the second wall is part of a closure and/or of a mask body.

39. The expiration system of claim 23, wherein the first wall forms a funnel shape together with a first collar, an upper surface of the first wall being at an angle A of from 0° to 90° to an inner surface of the first wall, and the second wall forms a funnel shape together with a second collar, wherein an underside of the second collar is at an angle B of from 0° to 90° to an outer surface of the second wall, angle A being equal to angle B.

40. The expiration system of claim 27, wherein the grooves have a substantially rectangular cross section, optionally with rounded corners and/or a superimposed pitch circle, and have a maximum width of 1.25 mm and a maximum depth of 1.25 mm.

41. A patient interface, wherein the patient interface comprises the expiration system of claim 23 and a ball joint.

42. The patient interface of claim 41, wherein the ball joint comprises at least one ball head and a joint bearing, the ball head being substantially constituted by a third wall of the expiration system which at least partially encloses the second flow path and the joint bearing being substantially formed by an inner surface of the second wall.
